(19) United States
(12) Patent Application Publication
(10) Pub. No.: US 2026/0055165 A1
(43) Pub. Date: Feb. 26, 2026

(54) MONOCLONAL ANTIBODY ISOLATION

(71) Applicant: Imperial College Innovations Limited, London (GB)

(72) Inventors: Konstantinos PASCHOS, London (GB); Robin SHATTOCK, London (GB)

(73) Assignee: Imperial College Innovations Limited, London (GB)

(21) Appl. No.: 19/102,799

(22) PCT Filed: Aug. 10, 2023
(86) PCT No.: PCT/GB2023/052116

§ 371 Date: Feb. 10, 2025

(30) Foreign Application Priority Data
Aug. 11, 2022 (GR) 20220100678

(51) Int. Cl. C07K 16/10; C12N 5/0781; C12N 15/86; C12P 21/02

(52) U.S. Cl. CPC C07K 16/1003; C12N 5/0635; C12N 15/86; C12P 21/02; C07K 2317/24; C07K 2317/76; C12N 2502/99; C12N 2710/16243; C12N 2740/15043

(57) ABSTRACT

The invention relates to monoclonal antibody production and isolation, and particularly, although not exclusively, to a novel feeder cell line for culturing a monoclonal antibody-producing B cell. The invention also extends to the use of the feeder cell line in culturing a monoclonal antibody-producing B cell, and isolating the B cell from the cell culture media. The invention further extends to methods for culturing and isolating a monoclonal antibody-producing B cell, as well as a method for isolating a monoclonal antibody.

The present invention relates to monoclonal antibody production and isolation, and particularly, although not exclusively, to a novel feeder cell line for culturing a monoclonal antibody-producing B cell. The invention also extends to the use of the feeder cell line in culturing a monoclonal antibody-producing B cell, and isolating the B cell from the cell culture media. The invention further extends to methods for culturing and isolating a monoclonal antibody-producing B cell, as well as a method for isolating a monoclonal antibody.

Epstein-Barr virus (EBV) is a B lymphotropic gammaherpes virus that asymptomatically and persistently infects over 90% of humans. In vitro, EBV induces the continuous proliferation (transformation and “immortalisation”) of human B cells to create lymphoblastoid cell lines (LCLs). EBV-infected LCLs that originate from memory B cells specific for an antigen, produce an antibody against this antigen. This EBV feature has been exploited in the past (1) to isolate human monoclonal antibodies (MABs).

However, there are several deficiencies with the current approach of using EBV to obtain MABs. Firstly, there is a relatively low efficiency of B cell transformation by EBV. This has been addressed to an extent by the addition of a 24-mer oligodeoxy-nucleotide, CpG ODN 2006, in culture media of EBV-infected cells. CpG ODN 2006 acts as a TLR9 agonist and increases transformation efficiency from ˜2% to ˜30% (2). Secondly, there exists a difficulty in growing monoclonal cultures to produce MABs. Isolated single LCL cells do not proliferate, meaning that cultures are grown first oligoclonally, adding several cells of different specificities to the same culture well. After two weeks of growth, culture supernatants are assessed for antibody production and the positive cultures are subcloned again by limiting dilution. After several weeks, the new subcloned cultures are further assessed to test for clonality, antibody production and specificity (3). Additionally, the current approach suffers from a low throughput of growing cultures and subcultures and assessing growth or clonality.

In view of the above problems associated with the classic EBV method, an alternative method of single cell molecular cloning is currently the preferred option for isolating MABs (4). This method involves single cell sorting of antigen specific B cells by FACS, followed by molecular cloning from single cells, which determines the antibody sequence for each cell. The sequence information is used to construct plasmid expression vectors for antibody production in separate cells, specialised for protein (antibody) expression. The antibodies can be assessed for specificity, affinity, and neutralisation after antibody production from these protein expression specialised cells. This is a relatively high throughput method that reduces the time required to obtain MABs by several weeks, compared to the classic EBV method, and overall efficiency is almost double (5).

However, there are also a number of disadvantages associated with single cell molecular cloning and antibody production from expression-specialised mammalian cells. Firstly, obtaining sequence information, performing molecular cloning, and expressing antibodies in separate cells is significantly more expensive than culturing EBV-infected, antibody producing LCLs. Secondly, obtaining antibody sequence information from single cells is challenging and compromises efficiency. Finally, the testing of antibodies for specificity, affinity, and neutralisation is not possible before expression from plasmid vectors in expression-specialised cell lines.

There is, therefore, a need to provide an improved platform for isolating human monoclonal antibodies, with increased efficiency and high throughput capability.

As described in the Examples, the inventors have developed a new platform for isolating human monoclonal antibodies from human peripheral blood mononuclear cells (PBMCs). In particular, the inventors' have developed a new feeder cell line, called Amalthea, and have surprisingly demonstrated that this feeder cell line can be used to support efficient outgrowth of antibody-producing B cells that are infected with a recombinant EBV. The inventors have demonstrated that the Amalthea feeders can support monoclonal cell cultures of antibody-producing cells, and the MABs produced in the media can be used in multiple assays straight away, significantly saving time and lowering cost. Additionally, the platform is compatible with fluorescence activated cell sorting (FACS), used for the selection of B cells specific for an antigen of interest, massively increasing throughput.

Therefore, according to a first aspect of the invention, there is provided a feeder cell line for culturing a monoclonal antibody-producing B cell, the feeder cell line expressing:
mega CD40 ligand (mega CD40L), or a variant or fragment thereof;
CD23, or a variant or fragment thereof; and/or
a fluorescent protein that is not expressed by the B cell.

According to a second aspect of the invention, there is provided use of a feeder cell line in culturing a monoclonal antibody-producing B cell, wherein the feeder cell line expresses:
mega CD40 ligand (mega CD40L), or a variant or fragment thereof; and/or
CD23, or a variant or fragment thereof.

According to a third aspect of the invention, there is provided use of a feeder cell line in isolating a monoclonal antibody-producing B cell, wherein the feeder cell line expresses a fluorescent protein that is not expressed by the B cell.

According to a fourth aspect of the invention, there is provided use of a feeder cell line in isolating a monoclonal antibody-producing B cell, wherein the feeder cell line does not express a drug selection marker that is expressed by the B cell.

According to a fifth aspect of the invention, there is provided a method of culturing a monoclonal antibody-producing B cell, the method comprising:
(i) contacting a B cell with a feeder cell line expressing:
   mega CD40 ligand (mega CD40L), or a variant or fragment thereof; and/or
   CD23, or a variant or fragment thereof; and
(ii) culturing the B cell and the feeder cell line under conditions to support the growth of the monoclonal antibody-producing B cell.

According to a sixth aspect of the invention, there is provided a method of isolating a monoclonal antibody-producing B cell from a cell culture media, the method comprising:
(i) contacting a B cell with a feeder cell line expressing a fluorescent protein that is not expressed by the B cell; and
(ii) identifying the feeder cell line expressing the fluorescent protein to allow isolation of the monoclonal antibody-producing B cell.

According to a seventh aspect of the invention, there is provided a method of isolating a monoclonal antibody-producing B cell from a cell culture media, the method comprising:
(i) contacting a B cell with a feeder cell line, wherein the feeder cell line does not express a drug selection marker that is expressed by the B cell; and
(ii) culturing the B cell and the feeder cell line in the presence of the drug selection marker to allow isolation of the monoclonal antibody-producing B cell.

Preferably, the method of the fifth, sixth and seventh aspect comprises a step of isolating a monoclonal antibody from the monoclonal antibody-producing B cell.

Hence, according to an eighth aspect of the invention, there is provided a method of isolating a monoclonal antibody from a monoclonal antibody-producing B cell, the method comprising:
(i) contacting a B cell with a feeder cell line expressing:
   mega CD40 ligand (mega CD40L), or a variant or fragment thereof;
   CD23, or a variant or fragment thereof; and/or
   a fluorescent protein that is not expressed by the B cell,
(ii) culturing the B cell and the feeder cell line under conditions to support the growth of the monoclonal antibody-producing B cell, and
(iii) isolating a monoclonal antibody from the monoclonal antibody-producing B cell.

As described in Examples 2 and 3, the inventors have demonstrated that modifying a feeder cell line to express mega CD40L and/or CD23, results in a feeder cell line that can significantly increase the number of outgrowing monoclonal antibody-producing B cells. For example, the inventors have demonstrated that their feeder cell line increases virus-mediated transformation/immortalisation efficiency of B cells by more than 4-fold. Additionally, with the inventors' new feeder cell line, it is now possible to support monoclonal outgrowth of virus-infected cells for the first time without sub-cloning steps and serial dilutions, saving several weeks compared to the classic EBV method for monoclonal antibody isolation and greatly increasing efficiency. Furthermore, the resulting monoclonal cultures' supernatants contain antibody that can be assessed without the need for molecular cloning of the antibody sequence, which is required for the method most favoured currently. This brings down the cost per antibody assessed 50− to 100− fold, relative to the single-cell molecular cloning method currently preferred.

Furthermore, as described in Example 4, by also modifying the feeder cell line to express a fluorescent protein that is not expressed by the antibody-producing B cell, the inventors have demonstrated that it is possible to readily distinguish the feeder cells from the B cells, without any additional staining steps that would result in the loss of cells of interest.

In one embodiment, the feeder cell line expresses: (i) mega CD40L, or a variant or fragment thereof; and (ii) CD23, or a variant or fragment thereof. Optionally, the feeder cell line also expresses: (iii) a fluorescent protein that is not expressed by the B cell.

In another embodiment, the feeder cell line expresses: (i) mega CD40L, or a variant or fragment thereof; and (ii) a fluorescent protein that is not expressed by the B cell. Optionally, the feeder cell line also expresses: CD23, or a variant or fragment thereof.

In another embodiment, the feeder cell line expresses: (i) CD23, or a variant or fragment thereof; and (ii) a fluorescent protein that is not expressed by the B cell. Optionally, the feeder cell line also expresses: mega CD40L, or a variant or fragment thereof.

Alternatively, in another embodiment, the feeder cell line expresses: (i) mega CD40L, or a variant or fragment thereof; (ii) CD23, or a variant or fragment thereof; and (iii) a fluorescent protein that is not expressed by the B cell.

As used herein, the term “feeder cell line” can refer to a line of cells that are used in a culture of target cells (i.e. B cells), to support their survival and/or growth, for example, by producing various growth factors. Additionally, the term “feeder cell line” can encompass cells which have been engineered to express particular growth factors or proteins, such as mega CD40L and/or CD23.

The feeder cell line may be any cell line capable of supporting the growth of a monoclonal antibody-producing B cell. For example, the feeder cell line may be selected from a group consisting of an osteosarcoma cell line, a mesenchymal cell line, an epithelial cell line, a lymphoblastoid cell line, a neuronal cell line and/or an endothelial cell line. Most preferably, the feeder cell line is an osteosarcoma cell line.

In one embodiment, the feeder cell line may be selected from a group consisting of U2OS, MRC5, lymphoblastoid cell line (LCL), H1299, MCF7, HEK293, 3T3, Caco-2, and/or HeLa. U2OS is a cell line with epithelial morphology, derived from the bone tissue of an osteosarcoma patient. MRC5 is a diploid cell line composed of human fibroblast cells, derived from lung tissue. H1299 is a human non-small cell lung carcinoma cell line derived from the lymph node. MCF7 is an epithelial cell line derived from human breast cancer cells. HEK293 is a cell line with epithelial morphology, derived from the kidney of a human embryo. 3T3 is a fibroblast cell line that was isolated from the embryo of a mouse. Caco-2 is an epithelial cell line, derived from a colon carcinoma. HeLA is an immortal cell line, originally isolated from a cervical carcinoma. Most preferably, the feeder cell line is U2OS. Advantageously, this cell line is adherent and forms a feeder monolayer for LCL cells (i.e. the monoclonal antibody-producing B cells).

In one embodiment, the feeder cell line is irradiated. Preferably, the feeder cell line is irradiated prior to contacting the B cell with the feeder cell line. Advantageously, irradiation arrests growth of the feeder cells and prevents them from taking over the culture.

In one embodiment, the feeder cell line is irradiated using a ¹³⁷Cs γ-ray irradiator. Preferably, the feeder cell line is irradiated using a ¹³⁷Cs γ-ray irradiator at a radiation dose of at least 5 Gy, at least 10 Gy, at least 15 Gy, at least 20 Gy or at least 25 Gy. More preferably, the feeder cell line is irradiated using a 137Cs y-ray irradiator at a radiation dose of at least 30 Gy.

In one embodiment, the feeder cell line is irradiated using an X-ray irradiator. Alternatively, in another embodiment, the feeder cell line is not irradiated. Instead, the feeder cell line may be treated transiently with cell cycle inhibiting drugs. In one embodiment, the cell cycle inhibiting drug is Mitomycin C. Alternatively, in another embodiment, the feeder cell line is not irradiated or treated with cell cycle inhibiting drugs.

CD40 is a TNF superfamily type II transmembrane protein. CD40 ligand (CD40L) is the physiological ligand that binds to CD40 on the surface of B cells and provides an activation and survival signal for LCL outgrowth (6,7). Mega CD40L consists of two trimers of CD40L, and advantageously, mega CD40L is more stable and mitogenic than physiological CD40L (7).

One embodiment of the polypeptide sequence of mega CD40L is represented herein as SEQ ID No: 1, as follows:

[SEQ ID No: 1]
MKANLLVLLCALAAADADYKDDDDKGPGQVQLHEDDVTTTEELAPAL
VPPPKGTCAGWMAGIPGHPGHNGTPGRDGRDGTPGEKGEKGDAGLLG
PKGETGDVGMTGAEGPRGFPGTPGRKGEPGELQGDQNPQIAAHVISE
ASSKTTSVLQWAEKGYYTMSNNLVTLENGKQLTVKRQGLYYIYAQVT
FCSNREASSQAPFIASLCLKSPGRFERILLRAANTHSSAKPCGQQSI
HLGGVFELQPGASVFVNVTDPSQVSHGTGFTSFGLLKL

Preferably, therefore, mega CD40L comprises an amino acid sequence substantially as set out in SEQ ID No: 1, or a fragment or variant thereof.

In one embodiment, the nucleotide sequence encoding mega CD40L is represented to herein as SEQ ID No: 2, as follows:

[SEQ ID No: 2]
ATGAAGGCCAACCTGCTGGTGCTGCTGTGTGCTCTGGCTGCCGCCGA
TGCCGACTACAAGGACGACGATGACAAAGGCCCTGGACAGGTCCAGC
TGCACGAGGATGATGTGACCACCACCGAAGAACTGGCCCCTGCTCTT
GTGCCTCCTCCAAAGGGAACATGTGCCGGCTGGATGGCTGGAATCCC
TGGACACCCAGGCCACAATGGCACACCTGGCAGAGATGGAAGAGATG
GCACCCCAGGCGAGAAGGGCGAAAAAGGCGACGCTGGACTGCTGGGA
CCTAAAGGCGAAACTGGCGACGTGGGAATGACAGGCGCTGAGGGCCC
TAGAGGCTTTCCTGGAACACCTGGAAGAAAGGGCGAGCCTGGCGAAC
TGCAGGGCGATCAGAATCCTCAGATTGCCGCTCACGTGATCAGCGAG
GCCAGCAGCAAGACAACAAGCGTGCTGCAGTGGGCCGAGAAGGGGTA
CTACACCATGAGCAACAACCTGGTCACCCTGGAAAACGGCAAGCAGC
TGACCGTGAAGAGACAGGGCCTGTACTACATCTACGCCCAAGTGACC
TTCTGCAGCAACAGAGAGGCCAGCTCTCAGGCCCCTTTTATCGCCAG
CCTGTGCCTGAAGTCCCCTGGCAGATTCGAGAGAATCCTGCTGAGAG
CCGCCAACACACACAGCTCCGCCAAACCTTGTGGCCAGCAGTCTATT
CACCTCGGCGGAGTGTTTGAGCTGCAGCCTGGCGCTAGCGTGTTCGT
GAATGTGACAGACCCTAGCCAGGTGTCCCACGGCACCGGCTTTACAT
CTTTCGGCCTGCTGAAGCTGTGA

Preferably, therefore, mega CD40L is encoded by a nucleotide sequence substantially as set out in SEQ ID No: 2, or a fragment or variant thereof.

Mega CD40L is available commercially as a soluble protein to be added to culture media and has been used previously to enhance LCL outgrowth (8,9). However, the feeder cell line according to the invention expresses mega CD40L directly into the culture, which advantageously creates a microenvironment with exceptionally high concentrations of the more active recombinant mega CD40L for the outgrowing cells to rest on. For example, the inventors surprisingly discovered that the feeder cell line according to the invention was able to achieve 4-7 times higher concentration of mega CD40L in the media microenvironment (see FIG. 2) compared to what is commonly added as a soluble protein (8,9). Furthermore, the inventors believe that the concentration of mega CD40L is even higher in the microenvironment close to the secreting feeders' cell surface, where the outgrowing LCL cells (i.e. the monoclonal antibody-producing B cells) rest.

As described herein, the monoclonal antibody-producing B cell is cultured with the feeder cell line in a cell culture media. As such, the mega CD40L expressed by the feeder cell line is expressed directly into the cell culture media, resulting in a particular concentration of megaCD40L in the cell culture media. Advantageously, the feeder cell line according to the invention expresses a high concentration of megaCD40L, or a variant or fragment thereof, which supports the growth of the monoclonal antibody-producing B cell.

Accordingly, in a preferred embodiment, the feeder cell line expresses at least 1 ng/ml, at least 2 ng/ml, at least 3 ng/ml, at least 4 ng/ml, or at least 5 ng/ml of mega CD40L, or a variant or fragment thereof, in the cell culture media. More preferably, the feeder cell line expresses at least 10 ng/ml, at least 20 ng/ml, at least 30 ng/ml, at least 40 ng/ml, or at least 50 ng/ml of mega CD40L, or a variant or fragment thereof, in the cell culture media. Even more preferably, the feeder cell line expresses at least 51 ng/ml of mega CD40L, or a variant or fragment thereof, in the cell culture media.

In another preferred embodiment, the feeder cell line expresses at least 60 ng/ml, at least 70 ng/ml, at least 80 ng/ml, at least 90 ng/ml, or at least 100 ng/ml of mega

CD40L, or a variant or fragment thereof, in the cell culture media. More preferably, the feeder cell line expresses at least 101 ng/ml of mega CD40L, or a variant or fragment thereof, in the cell culture media.

In another preferred embodiment, the feeder cell line expresses at least 110 ng/ml, at least 120 ng/ml, at least 130 ng/ml, at least 140 ng/ml, at least 150 ng/ml, at least 160 ng/ml, at least 170 ng/ml, at least 180 ng/ml, at least 190 ng/ml, or at least 200 ng/ml of mega CD40L, or a variant or fragment thereof, in the cell culture media. Even more preferably, the feeder cell line expresses at least 220 ng/ml, at least 240 ng/ml, at least 260 ng/ml, at least 280 ng/ml, or at least 300 ng/ml of mega CD40L, or a variant or fragment thereof, in the cell culture media. Even more preferably, the feeder cell line expresses at least 320 ng/ml, at least 340 ng/ml, at least 360 ng/ml, at least 380 ng/ml, or at least 400 ng/ml of mega CD40L, or a variant or fragment thereof, in the cell culture media.

CD23 is a low affinity IgE receptor present on the B cell membrane, upregulated by IL-4. CD23 is a 45-kDa type II membrane glycoprotein isoform, i.e. two isoforms exist, CD23a and CD23b.

Accordingly, in one embodiment, the CD23 or fragment or variant thereof, expressed by the feeder cell line, is CD23a or CD23b.

CD23a and CD23b act as an LCL growth factor, and both isoforms have the same effect as they result in the same domain being presented on the surface of the feeder cells (10,11). CD23b is specifically expressed by LCLs and only LCL cells expressing CD23 are transformed. LCL transformation is enhanced at early stages by proximity to other CD23-expressing LCLs (12,13). However, previous methods have either concentrated on CD23 soluble fragment that is shed from B cells (10), or on the full protein extracted by cell lysis and then used as an additive in cell culture (11).

In contrast, the feeder cell line according to the invention was made to express full length CD23 to enhance single cell LCL cloning, in the absence of other LCL cells. Advantageously, this facilitates efficient monoclonal outgrowth following single-cell index sorting for the establishment of monoclonal LCL cultures. The inventors surprisingly found that feeder cell lines expressing CD23b increased LCL outgrowth (i.e. the monoclonal antibody-producing B cells) by ˜20%.

Most preferably, therefore, the feeder cell line expresses CD23b.

One embodiment of the polypeptide sequence of CD23b is represented herein as SEQ ID No: 3, as follows:

[SEQ ID No: 3]
MNPPSQEIEELPRRRCCRRGTQIVLLGLVTAALWAGLLTLLLLWHWD
TTQSLKQLEERAARNVSQVSKNLESHHGDQMAQKSQSTQISQELEEL
RAEQQRLKSQDLELSWNLNGLQADLSSFKSQELNERNEASDLLERLR
EEVTKLRMELQVSSGFVCNTCPEKWINFQRKCYYFGKGTKQWVHARY
ACDDMEGQLVSIHSPEEQDFLTKHASHTGSWIGLRNLDLKGEFIWVD
GSHVDYSNWAPGEPTSRSQGEDCVMMRGSGRWNDAFCDRKLGAWVCD
RLATCTPPASEGSAESMGPDSRPDPDGRLPTPSAPLHS

Preferably, therefore, CD23b comprises an amino acid sequence substantially as set out in SEQ ID No: 3, or a variant or fragment thereof.

In one embodiment, the nucleotide sequence encoding CD23b is represented herein as SEQ ID No: 4, as follows:

[SEQ ID No: 4]
ATGAATCCTCCAAGCCAAGAGATCGAGGAACTGCCCCGCAGACGGTG
CTGTAGAAGAGGCACACAGATCGTGCTGCTGGGCCTTGTGACAGCTG
CTCTGTGGGCTGGACTGCTGACACTGCTGCTGCTGTGGCACTGGGAT
ACCACACAGAGCCTGAAGCAGCTGGAAGAAAGGGCCGCCAGAAACGT
GTCCCAGGTGTCCAAGAACCTGGAAAGCCACCACGGCGACCAGATGG
CCCAGAAGTCTCAGAGCACCCAGATCAGCCAAGAGCTTGAAGAACTG
AGAGCCGAGCAGCAGCGGCTGAAGTCCCAAGATCTGGAACTGAGCTG
GAACCTGAACGGACTGCAGGCCGATCTGAGCAGCTTCAAGTCTCAAG
AGCTGAACGAGAGAAACGAGGCCAGCGACCTGCTGGAACGGCTGAGA
GAAGAAGTGACCAAGCTGCGGATGGAACTGCAGGTTTCCAGCGGCTT
CGTGTGCAACACATGCCCCGAGAAGTGGATCAACTTCCAGCGGAAGT
GCTACTACTTCGGCAAGGGCACCAAGCAGTGGGTGCACGCCAGATAC
GCCTGCGACGATATGGAAGGCCAGCTGGTGTCCATTCACAGCCCCGA
GGAACAGGACTTCCTGACCAAACACGCCAGCCACACCGGCTCTTGGA
TCGGACTGAGAAACCTGGACCTGAAGGGCGAGTTCATCTGGGTGGAC
GGCAGCCACGTGGACTACTCTAATTGGGCTCCTGGCGAGCCCACCAG
CAGATCTCAAGGCGAGGATTGCGTGATGATGAGAGGCAGCGGCAGAT
GGAACGACGCCTTCTGCGATAGAAAGCTCGGCGCCTGGGTTTGCGAC
AGACTGGCCACATGTACACCTCCAGCCTCTGAGGGAAGCGCCGAGTC
TATGGGCCCTGACTCTAGACCCGATCCTGACGGCAGACTGCCTACAC
CTTCTGCTCCTCTGCACAGCTGA

Preferably, therefore, CD23b is encoded by a nucleotide sequence substantially as set out in SEQ ID No: 4, or a fragment or variant thereof.

The feeder cell line may express CD23a.

One embodiment of the polypeptide sequence of CD23a is represented herein as SEQ ID No: 5, as follows:

[SEQ ID No: 5]
MEEGQYSEIEELPRRRCCRRGTQIVLLGLVTAALWAGLLTLLLLWHW
DTTQSLKQLEERAARNVSQVSKNLESHHGDQMAQKSQSTQISQELEE
LRAEQQRLKSQDLELSWNLNGLQADLSSFKSQELNERNEASDLLERL
REEVTKLRMELQVSSGFVCNTCPEKWINFQRKCYYFGKGTKQWVHAR
YACDDMEGQLVSIHSPEEQDFLTKHASHTGSWIGLRNLDLKGEFIWV
DGSHVDYSNWAPGEPTSRSQGEDCVMMRGSGRWNDAFCDRKLGAWVC
DRLATCTPPASEGSAESMGPDSRPDPDGRLPTPSAPLHS

Preferably, therefore, CD23a comprises an amino acid sequence substantially as set out in SEQ ID No: 5, or a variant or fragment thereof.

In one embodiment, the codon optimized nucleotide sequence encoding CD23a is represented herein as SEQ ID No: 6, as follows:

[SEQ ID No: 6]
atggaagagggccagtacagcgagatcgaggaactgcctcggcggag
atgctgtagaagaggcacacagatcgtgctgctgggccttgtgacag
ctgctctgtgggctggactgctgacactgctgctgctgtggcactgg
gataccacacagagcctgaagcagctggaagaaagggccgccagaaa
cgtgtcccaggtgtccaagaacctggaaagccaccacggcgaccaga
tggcccagaagtctcagagcacccagatcagccaagagcttgaggaa
ctgagagccgagcagcagagactgaagtcccaggacctggaactgag
ctggaacctgaatggactgcaggccgacctgagcagcttcaagtccc
aagagctgaacgagagaaacgaggccagcgacctgctggaacggctg
agagaagaagtgaccaagctgcggatggaactgcaggtttccagcgg
cttcgtgtgcaacacatgccccgagaagtggatcaacttccagcgga
agtgctactacttcggcaagggcaccaagcagtgggtgcacgccaga
tacgcctgcgacgatatggaaggccagctggtgtccattcacagccc
cgaggaacaggacttcctgaccaaacacgccagccacaccggctctt
ggatcggcctgagaaacctggatctgaagggcgagttcatctgggtg
gacggcagccacgtggactactctaattgggctcctggcgagcccac
cagcagatctcaaggcgaggattgcgtgatgatgagaggcagcggca
gatggaacgacgccttctgcgatagaaagctcggcgcctgggtttgc
gacagactggccacatgtacacctccagcctctgagggaagcgccga
gtctatgggccctgactctagacccgatcctgacggcagactgccta
caccttctgctcctctgcacagctga

Preferably, therefore, CD23a is encoded by a nucleotide sequence substantially as set out in SEQ ID No: 6, or a fragment or variant thereof.

The inventors have demonstrated that the expression of a fluorescent protein in the feeder cell line, makes the feeder cells easily distinguishable from the LCLs (i.e. the monoclonal antibody-producing B cells). As such, unlike with the prior art methods, by using the feeder cell line of the invention, it is therefore not necessary to stain the new LCLs before FACs for single B cell cloning, even if a non-recombinant, non-GFP expressing EBV is used. This is significantly advantageous, given that additional staining steps would result in loss of the target B cells.

Accordingly, in one embodiment, the feeder cell line expresses a fluorescent protein that is not expressed by the B cell.

In some embodiments, the monoclonal antibody-producing B cell is cultured using Epstein Barr Virus (EBV).

The EBV may be selected from any variant capable of inducing the continuous proliferation (transformation and “immortalisation”) of B cells. Thus, preferably the B cell is an EBV-infected monoclonal antibody-producing B cell. In some embodiments, a recombinant EBV is used. Thus, most preferably the B cell is a recombinant EBV-infected monoclonal antibody-producing B cell.

In one embodiment, the EBV expresses a drug selection marker. Preferably, the EBV expresses the hygromycin resistance gene. This allows hygromycin antibiotic selection of EBV-infected B cells (14). Advantageously, because the EBV-infected B cell expresses a drug selection marker (e.g. a hygromycin resistance gene), it is possible to kill the feeder cells without affecting the EBV-infected antibody-producing B cells. Otherwise, ¹³⁷Cs γ-ray or X-ray irradiation would have to be used to arrest feeder cell growth, which requires equipment that is not always available. Or alternatively, feeder cells would have to be transiently treated with cell cycle inhibitors such as Mitomycin C, which requires thorough washing, and leftover contaminants may affect the growth of the monoclonal antibody-producing B cells.

In one embodiment, the nucleotide sequence of the hygromycin resistance gene is represented herein as SEQ ID No: 7, as follows:

[SEQ ID No: 7]
atgaaaaagcctgaactcaccgcgacgtctgtcgagaagtttctgat
cgaaaagttcgacagcgtctccgacctgatgcagctctcggagggcg
aagaatctcgtgctttcagcttcgatgtaggagggcgtggatatgtc
ctgcgggtaaatagctgcgccgatggtttctacaaagatcgttatgt
ttatcggcactttgcatcggccgcgctcccgattccggaagtgcttg
acattggggaattcagcgagagcctgacctattgcatctcccgccgt
gcacagggtgtcacgttgcaagacctgcctgaaaccgaactgcccgc
tgttctgcagccggtcgcggaggccatggatgcgatcgctgcggccg
atcttagccagacgagcgggttcggcccattcggaccgcaaggaatc
ggtcaatacactacatggcgtgatttcatatgcgcgattgctgatcc
ccatgtgtatcactggcaaactgtgatggacgacaccgtcagtgcgt
ccgtcgcgcaggctctcgatgagctgatgctttgggccgaggactgc
cccgaagtccggcacctcgtgcacgcggatttcggctccaacaatgt
cctgacggacaatggccgcataacagcggtcattgactggagcgagg
cgatgttcggggattcccaatacgaggtcgccaacatcttcttctgg
aggccgtggttggcttgtatggagcagcagacgcgctacttcgagcg
gaggcatccggagcttgcaggatcgccgcggctccgggcgtatatgc
tccgcattggtcttgaccaactctatcagagcttggttgacggcaat
ttcgatgatgcagcttgggcgcagggtcgatgcgacgcaatcgtccg
atccggagccgggactgtcgggcgtacacaaatcgcccgcagaagcg
cggccgtctggaccgatggctgtgtagaagtactcgccgatagtgga
aaccgacgccccagcactcgtccgagggcaaaggaatag

Preferably, therefore, the hygromycin resistance gene comprises a nucleotide sequence substantially as set out in SEQ ID No: 7, or a fragment or variant thereof.

The EBV may be engineered so that its genome contains at least 1, 2, 3, 4, 5, or 6 3kb BamHI W repeat sequences, preferably with each repeat coding a W promoter. Advantageously, this increases transformation efficiency of infected B cells, and therefore, EBV clones that exhibited expansion of the repeat region to 6.6 repeats were selected for use (15,16).

The EBV may be engineered to express a fluorescent protein, which therefore becomes expressed by the infected B cell. Accordingly, in a preferred embodiment, the EBV expresses a fluorescent protein.

Accordingly, in one embodiment, the feeder cell line expresses a first fluorescent protein, and the B cell expresses a second fluorescent protein, wherein the first and second fluorescent proteins are different. The first and second fluorescent protein may be selected from any fluorescent protein well-known to the skilled person, for example, green fluorescent protein (GFP) and its variants, mCherry, mNeonGreen, monomeric red fluorescent protein (mRFP), monomeric Infrared Fluorescent Protein (mIFP), Venus, Tag Red Fluorescent Protein 657 (TagRFP657), monomeric Apple (mApple), monomeric Tag Blue Fluorescent Protein (mTagBFP2), tdTomato, and/or Enhanced Yellow Fluorescent Protein (EYFP).

Preferably, the first and second fluorescent proteins emit fluorescent signals at a different wavelength, and therefore, the groups of cells can be distinguished from one another.

Preferably, the EBV used to infect the B cell expresses green fluorescent protein (GFP). Even more preferably, the EBV used to infect the B cell expresses enhanced green fluorescent protein. Accordingly, in a preferred embodiment, the B cell expresses enhanced green fluorescent protein. This allows rapid detection of populations of EBV-infected B cells.

One embodiment of the polypeptide sequence of enhanced GFP is represented herein as SEQ ID No: 8, as follows:

[SEQ ID No: 8]
MVSKGEELFTGVVPILVELDGDVNGHKFSVSGEGEGDATYGKLTLKF
ICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHK
LEYNYNSHNVYIMADKQKNGIKVNFKIRHNIEDGSVQLADHYQQNTP
IGDGPVLLPDNHYLSTQSALSKDPNEKRDHMVLLEFVTAAGITLGMD
ELYKSGLRSRAQASNSAVDGTAGPGSTGSR*

Preferably, therefore, enhanced GFP comprises an amino acid sequence substantially as set out in SEQ ID No: 8, or a fragment or variant thereof.

In one embodiment, the nucleotide sequence encoding enhanced GFP is represented to herein as SEQ ID No: 9, as follows:

[SEQ ID No: 9]
Atggtgagcaagggcgaggagctgttcaccggggtggtgcccatcct
ggtcgagctggacggcgacgtaaacggccacaagttcagcgtgtccg
gcgagggcgagggcgatgccacctacggcaagctgaccctgaagttc
atctgcaccaccggcaagctgcccgtgccctggcccaccctcgtgac
caccctgacctacggcgtgcagtgcttcagccgctaccccgaccaca
tgaagcagcacgacttcttcaagtccgccatgcccgaaggctacgtc
caggagcgcaccatcttcttcaaggacgacggcaactacaagacccg
cgccgaggtgaagttcgagggcgacaccctggtgaaccgcatcgagc
tgaagggcatcgacttcaaggaggacggcaacatcctggggcacaag
ctggagtacaactacaacagccacaacgtctatatcatggccgacaa
gcagaagaacggcatcaaggtgaacttcaagatccgccacaacatcg
aggacggcagcgtgcagctcgccgaccactaccagcagaacaccccc
atcggcgacggccccgtgctgctgcccgacaaccactacctgagcac
ccagtccgccctgagcaaagaccccaacgagaagcgcgatcacatgg
tcctgctggagttcgtgaccgccgccgggatcactctcggcatggac
gagctgtacaagtccggactcagatctcgagctcaagcttcgaattc
tgcagtcgacggtaccgcgggcccgggatccaccggatctagataa

Preferably, therefore, enhanced GFP is encoded by a nucleotide sequence substantially as set out in SEQ ID No: 9, or a fragment or variant thereof.

In another preferred embodiment, the feeder cell line expresses mCherry. One embodiment of the polypeptide sequence of mCherry is represented herein as SEQ ID No: 10, as follows:

[SEQ ID No: 10]
MVSKGEEDNMAIIKEFMRFKVHMEGSVNGHEFEIEGEGEGRPYEGTQ
TAKLKVTKGGPLPFAWDILSPQFMYGSKAYVKHPADIPDYLKLSFPE
GFKWERVMNFEDGGVVTVTQDSSLQDGEFIYKVKLRGTNFPSDGPVM
QKKTMGWEASSERMYPEDGALKGEIKQRLKLKDGGHYDAEVKTTYKA
KKPVQLPGAYNVNIKLDITSHNEDYTIVEQYERAEGRHSTGGMDELY
K

Preferably, therefore, mCherry comprises an amino acid sequence substantially as set out in SEQ ID No: 10, or a fragment or variant thereof.

In one embodiment, the nucleotide sequence encoding mCherry is represented to herein as SEQ ID No: 11, as follows:

[SEQ ID No: 11]
ATGGTGAGCAAGGGCGAGGAGGATAACATGGCCATCATCAAGGAGTT
CATGCGCTTCAAGGTGCACATGGAGGGCTCCGTGAACGGCCACGAGT
TCGAGATCGAGGGCGAGGGCGAGGGCCGCCCCTACGAGGGCACCCAG
ACCGCCAAGCTGAAGGTGACCAAGGGTGGCCCCCTGCCCTTCGCCTG
GGACATCCTGTCCCCTCAGTTCATGTACGGCTCCAAGGCCTACGTGA
AGCACCCCGCCGACATCCCCGACTACTTGAAGCTGTCCTTCCCCGAG
GGCTTCAAGTGGGAGCGCGTGATGAACTTCGAGGACGGCGGCGTGGT
GACCGTGACCCAGGACTCCTCCCTGCAGGACGGCGAGTTCATCTACA
AGGTGAAGCTGCGCGGCACCAACTTCCCCTCCGACGGCCCCGTAATG
CAGAAGAAGACCATGGGCTGGGAGGCCTCCTCCGAGCGGATGTACCC
CGAGGACGGCGCCCTGAAGGGCGAGATCAAGCAGAGGCTGAAGCTGA
AGGACGGCGGCCACTACGACGCTGAGGTCAAGACCACCTACAAGGCC
AAGAAGCCCGTGCAGCTGCCCGGCGCCTACAACGTCAACATCAAGTT
GGACATCACCTCCCACAACGAGGACTACACCATCGTGGAACAGTACG
AACGCGCCGAGGGCCGCCACTCCACCGGCGGCATGGACGAGCTGTAC
AAGTAG

Preferably, therefore, mCherry is encoded by a nucleotide sequence substantially as set out in SEQ ID No: 11, or a fragment or variant thereof.

In one embodiment, the feeder cell line does not express a drug selection marker. Preferably, the feeder cell line does not express the gene for hygromycin resistance.

This is because the EBV-infected B cells express a drug selection marker (e.g. the gene for hygromycin resistance), making it possible to select against feeder cells after initial outgrowth of the LCLs. Advantageously, therefore, the use of the drug selection marker makes it possible to select against the feeder cells without the use of ¹³⁷Cs γ-ray or X-ray irradiation, which requires additional equipment, or without cell cycle inhibiting drug treatment, which may affect the growth of the monoclonal antibody-producing B cells. In other words, this allows practical killing of the feeder cells without affecting the outgrowing LCL cells.

In one embodiment, the method of the fifth to eighth aspects further comprises the step of isolating a B cell from a sample obtained from a subject, preferably before the B cell is infected by EBV. Preferably, the B cell is isolated from the sample before the B cell is contacted with the feeder cell line (i.e. before step (i) of the method according to the fourth fifth to sixth eighth aspects). Preferably, the method comprises isolating a B cell that is specific for an antigen of interest. For example, the antigen may be an HIV antigen (see Example 8) or a SARS-COV-2 antigen (see Example 9). As described in Examples 8 and 9, the inventors demonstrated that the methods of the invention can be effectively used to isolate MABs specific for HIV-1 Env immunogen, e.g. ConM and ConS, and SARS-COV-2.

As used herein, the term “B cell” can refer to any type of B cell or derivative thereof, capable of producing an antibody. For example, the B cell may be a B-lymphocyte, a plasma B cell, an effector B cell, an activated B cell, or a memory B cell. The B cell may be obtained from a human that has been immunised with an antigen, or who has developed an immune response to an antigen as a result of disease. Alternatively, the B cell may be obtained from an immunised naïve person, which has not previously been exposed to the antigen of interest.

The B cell may be isolated from any biological sample, for example, blood, bone marrow, spleen, or lymph nodes. Preferably, the B cell is isolated from a blood sample. More preferably, the B cell is isolated from peripheral blood mononuclear cells (PBMCs). The blood may be venous or arterial blood. Blood samples may be assayed immediately. Alternatively, the blood sample may be stored at low temperatures, for example in a fridge or even frozen before the method is conducted. Alternatively, the blood sample may be stored at room temperature, for example between 18 to 22 degrees Celsius, before the method is conducted. Isolated PBMCs or B cells from bone marrow, spleen, or lymph nodes can be stored at −80 degrees Celsius.

A “subject” may be any person with B cells.

The B cell may be isolated from the biological sample using methods well-known to the skilled person, for example, fractionation using antibody coated magnetic beads, magnetic-activated cell sorting (MACS), or fluorescence-activated cell sorting (FACS). Preferably, the method comprises isolating the B cell by FACS. FACS may be used with any appropriate panel of markers to select B cells that are specific for an antigen. Preferably, cells are kept at 4 degrees Celsius before EBV infection.

The B cell may then be infected with EBV, causing it to become immortalised, i.e. divide and proliferate indefinitely. Accordingly, in one embodiment, the method further comprises the step of infecting the B cell with EBV. Preferably, the B cell is infected with EBV prior to being contacted with the feeder cell line (i.e. before step (i) of the method according to the fifth to eighth aspects). Even more preferably, the B cell is infected with EBV after the B cell has been isolated from a sample obtained from a subject. Preferably, the EBV is as described above.

In one embodiment, infecting the B cell with EBV comprises contacting the B cell with an EBV-containing RPMI media. Such media may be supplemented with foetal bovine serum (FBS, e.g. 10%), and preferably precleared by centrifugation (e.g. at about 2000 xg) and preferably filtered (e.g. through a 0.45 μm filter). Preferably, the B cell is contacted with the EBV-containing media at multiplicity of infection (MOI) of at least 10, at least 20, at least 30, or at least 40. More preferably, the B cell is contacted with the EBV-containing media at multiplicity of infection (MOI) of at least 50. Preferably, the B cell and EBV-containing media are incubated at about 37° C. with 5% CO₂. Even more preferably, the B cell and the EBV-containing media are incubated for at least one, at least two, at least three hours, or at least for four hours.

The method may further comprise the step of washing and re-suspending the B cell and the EBV mixture with Roswell Park Memorial Institute Medium (RPMI) media, preferably with FBS, most preferably 20% FBS. Preferably, the B cell and EBV mixture is washed with RPMI before the B cell is contacted with the feeder cell line (i.e. before step (i) of the method according to the fifth to eighth aspects). Preferably, the RPMI media is RPMI 1640 Medium, GlutaMAX™ Supplement (ThermoFisher, 61870143).

The method then comprises the step of contacting the B cell with the feeder cell line according to the invention (i.e. step (i) of the fifth to eighth aspects). Preferably, the feeder cell line is as described above. The B cell and feeder cell line are then cultured under conditions to support the growth of the monoclonal antibody-producing B cell (i.e. step (ii) of the fifth and eighth aspects). Therefore, it will be appreciated that the method produces a culture of monoclonal antibody-producing B cells. For example, the method may comprise contacting the B cell with RPMI media, preferably with FBS, more preferably with 20% FBS.

The method may further comprise contacting the B cell with a CpG oligonucleotide, preferably after the B cell has been contacted with the feeder cell line (i.e. after step (i) of the fifth to eighth aspects). CpGs are unmethylated DNA oligonucleotides, which act as a polyclonal activator of B cells. Preferably, the CpG oligonucleotide is a nuclease resistant phosphorothioate oligonucleotide. Preferably, the CpG oligonucleotide is CpG ODN 2006. One embodiment of the nucleotide sequence of CpG ODN 2006 is provided herein as SEQ ID No: 12, as follows:

[SEQ ID No: 12]
5′-tcgtcgttttgtcgttttgtcgtt-3′

Preferably, therefore, the CpG oligonucleotide comprises a nucleotide sequence substantially as set out in SEQ ID No: 12, or a fragment or variant thereof.

In one embodiment, the method comprises contacting the B cell with at least 0.2 μg/ml, at least 0.4 μg/ml, at least 0.6 μg/ml, at least 0.8 μg/ml, or at least 1.0 μg/ml of the CpG oligonucleotide. In another embodiment, the method comprises contacting the B cell with at least 1.2 μg/ml, at least 1.4 μg/ml, at least 1.6 μg/ml, at least 1.8 μg/ml, or at least 2.0 μg/ml of the CpG oligonucleotide. In a preferred embodiment, the method comprises contacting the B cell with at least 2.1 μg/ml, at least 2.2 μg/ml, at least 2.3 μg/ml, or at least 2.4 μg/ml of the CpG oligonucleotide. Most preferably, the method comprises contacting the B cell with at least 2.5 μg/ml CpG oligonucleotide.

In one embodiment, the method comprises culturing the B cell for at least two, at least three, at least four, at least five, or at least six days, most preferably, for at least seven days, preferably after the B cell has been contacted with the feeder cell line (i.e. after step (i) of the method according to the fifth and eighth aspects).

The method may then comprise isolating the monoclonal antibody-producing B cell (i.e. step (ii) of the sixth and seventh aspect). Preferably, the method according to the fifth and eighth aspects also comprises isolating the monoclonal antibody-producing B cell after step (ii). As described herein, the feeder cell line expresses a fluorescent protein that is not expressed by the B cell. Accordingly, the method may comprise identifying the feeder cell line expressing the fluorescent protein to allow isolation of the monoclonal antibody-producing B cell. The method may comprise isolating the monoclonal antibody-producing B cell using FACS.

The method may further comprise culturing the monoclonal antibody-producing B cell with RPMI, preferably with FBS, most preferably with 20% FBS. Preferably, the monoclonal antibody-producing B cell is cultured with RPMI after isolating the monoclonal antibody-producing B cell, i.e. step (ii) of the fifth aspect. The monoclonal antibody-producing B cell may also be cultured with a CpG oligonucleotide (preferably ODN 2006) at a concentration of at least 2.0 μg/ml, at least 2.5 μg/ml, at least 3 μg/ml, at least 3.5 μg/ml, at least 4.0 μg/ml, or at least 4.5 μg/ml. More preferably, the monoclonal antibody-producing B cell is cultured with a CpG oligonucleotide (preferably ODN 2006) at a concentration of at least 5.0 μg/ml. Preferably, the cultures are fed twice a week with fresh media. Even more preferably, the media of the first feed 25 only comprises CpG oligonucleotide (preferably ODN 2006). This step results in an expanded monoclonal B cell culture.

The expanded monoclonal B cell culture may then be assessed by the presence of fluorescently-labelled EBV infected cells. The assessment may be carried out after one or two weeks, for example detecting the presence of fluorescently-labelled EBV infected cells under a fluorescence microscope. The expanded B cell culture produces a monoclonal antibody, which preferably is able to bind to a particular antigen. As used herein, the term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts.

Accordingly, the method may comprise isolating the monoclonal antibody from the monoclonal antibody-producing B cell culture, i.e. step (iii) of the eighth aspect. Isolating the monoclonal antibody from the monoclonal antibody-producing B cell culture may comprise harvesting, centrifuging and/or filtering the cell culture media to obtain a cell culture supernatant comprising a monoclonal antibody. The method may further comprise separating and purifying the monoclonal antibody from the cell culture supernatant.

Advantageously, the feeder cell line according to the invention enables the culture of monoclonal antibody-producing B cells indefinitely, and preferably to a point where enough antibody is available in the culture supernatant for multiple assays, without the need for molecular cloning and expression in secondary protein-expression specialised cell lines.

Additionally, the feeder cell line according to the invention supports monoclonal culture to the point where it becomes self-sustaining indefinitely, and ideally to a point where thousands or even millions of identical antibody-producing cells are present in a single culture that can be harvested for efficient determination of the antibody sequence by molecular biology methods, far surpassing the efficiency of molecular cloning from a single cell, as is performed with the current methods. Accordingly, in a preferred embodiment, the method comprises obtaining the genetic sequence of the monoclonal antibody from mRNA produced by B cells in the monoclonal culture (as described in Example 8).

The method may be an in vitro or ex vivo method. Preferably, the method is an in vitro method.

As described herein, the feeder cell line according to the invention was obtained by transducing a starter cell line (preferably U2OS cells) with one or more expression vectors encoding megaCD40L, CD23b and a fluorescent marker, such as mCherry. Accordingly, the inventors have developed novel expression vectors, which may be used to transduce the feeder cell line according to the invention.

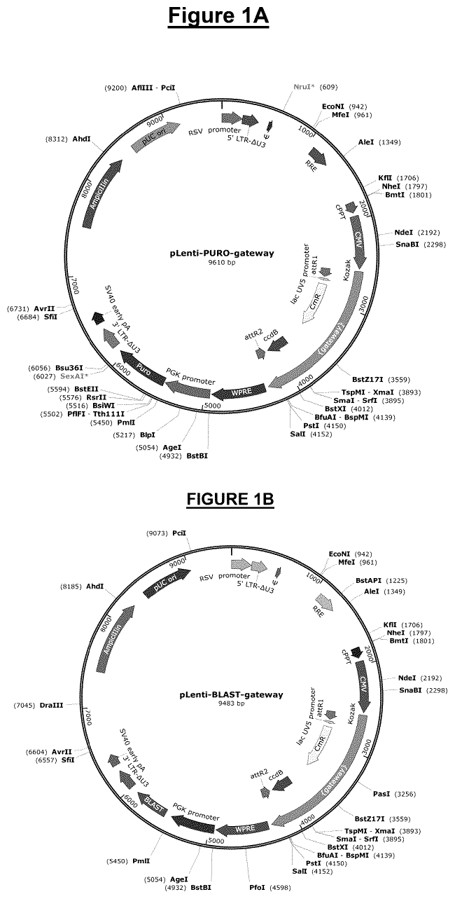

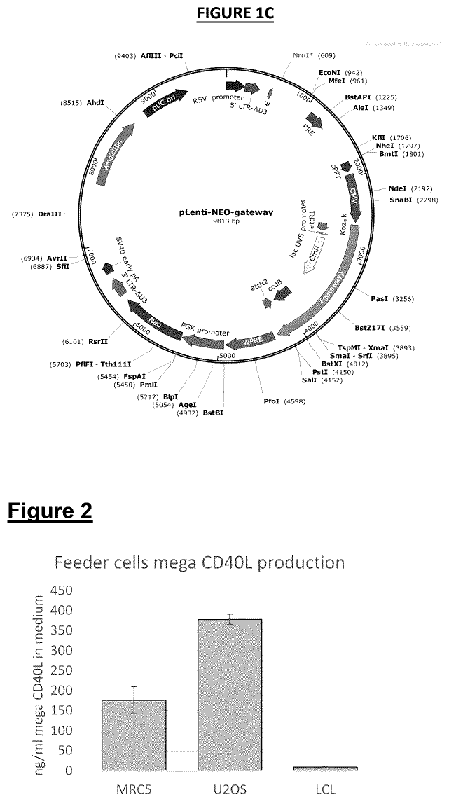

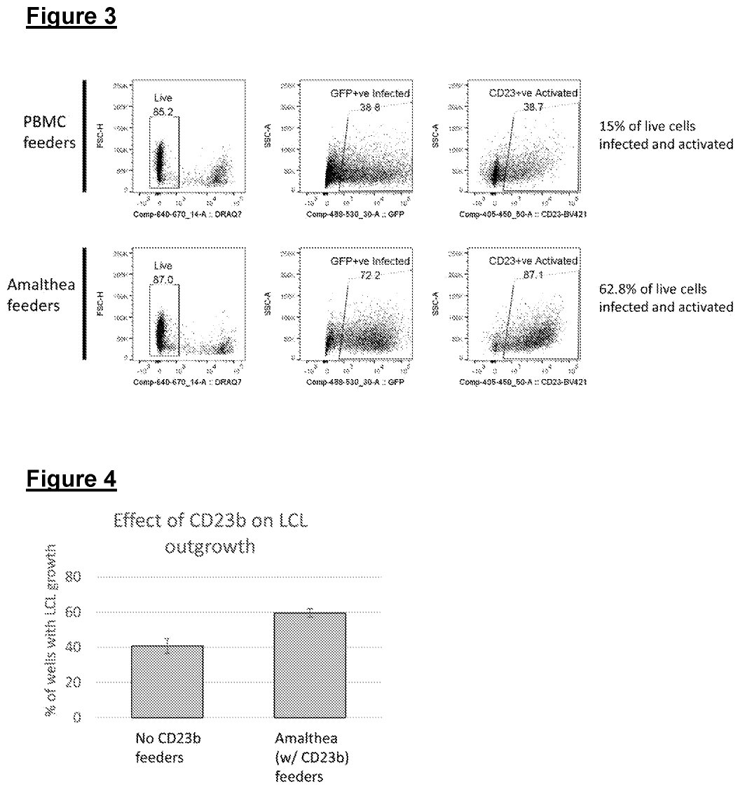

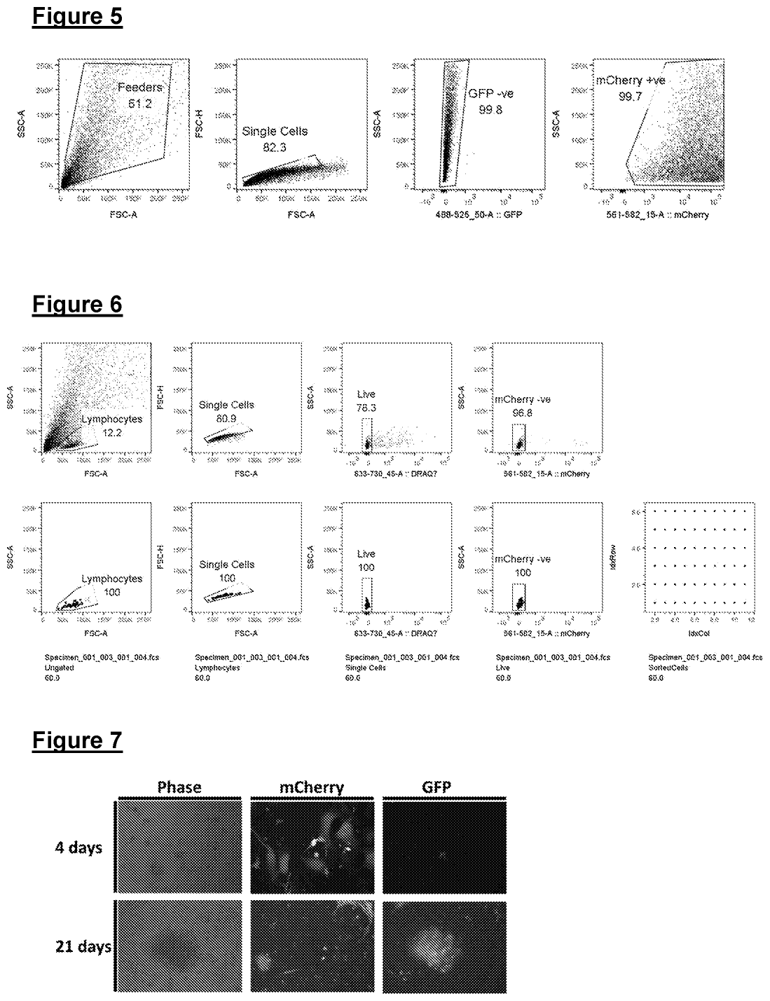

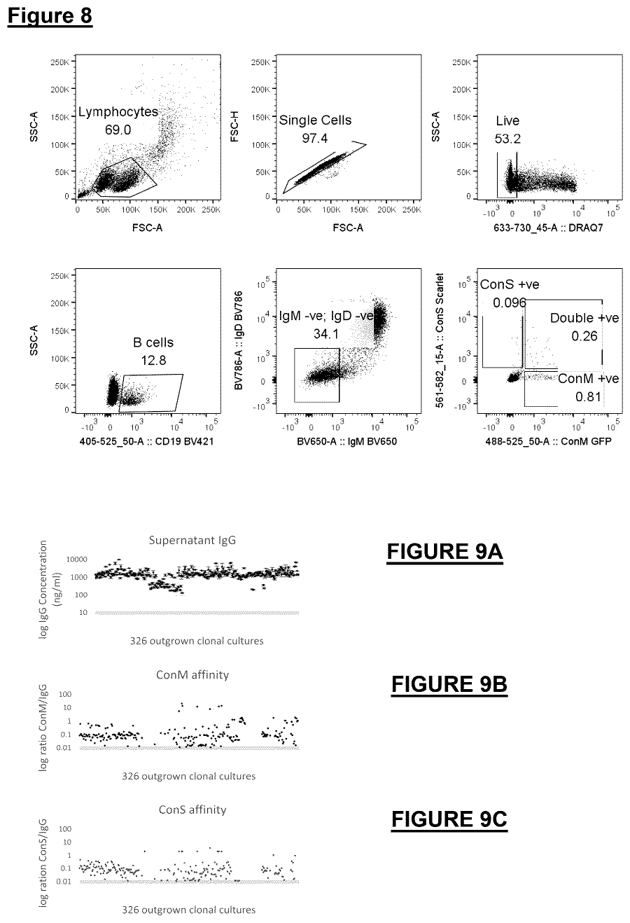

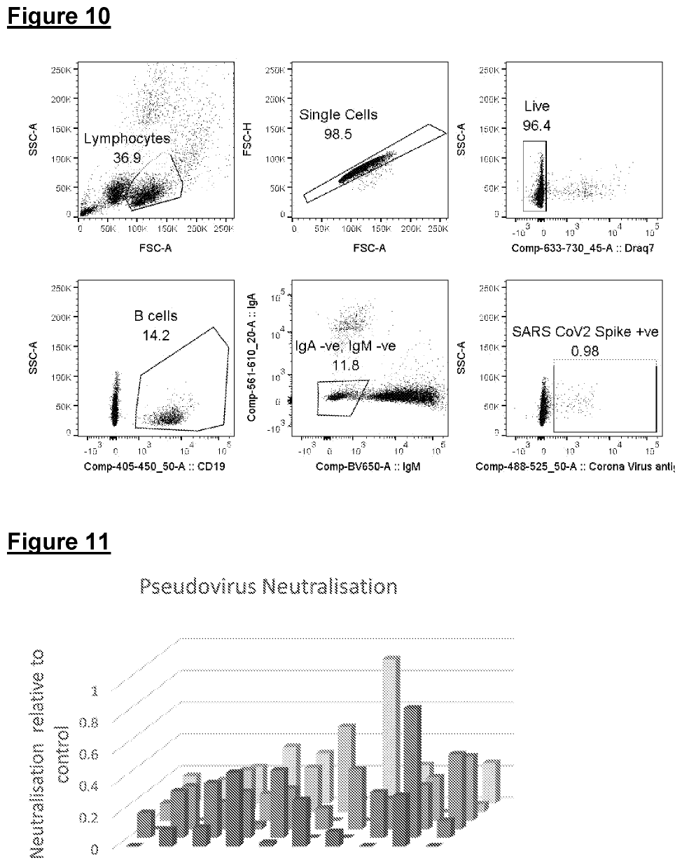

Hence, in a ninth aspect, there is provided a lentiviral vector substantially as illustrated in FIG. 1A, 1B or 1C.

In a tenth aspect, there is provided a kit for culturing a monoclonal antibody-producing B cell, the kit comprising:
(i) a feeder cell line expressing:
   (a) mega CD40 ligand (mega CD40L), or a variant or fragment thereof;
   (b) CD23, or a variant or fragment thereof; and/or
   (c) a fluorescent protein that is not expressed by the B cell.

Preferably, the kit is used to perform the method of any one of the fifth to eighth aspects.

In one embodiment, the kit further comprises EBV. In another embodiment, the kit further comprises reagents for isolating B cells from a biological sample. The EBV and reagents are as defined in the previous aspects of the invention.

The kit may further comprise instructions for use and/or a receptacle for obtaining a biological sample from a subject.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with any of the sequence identified herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:-(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and Tis the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:-Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2× SSC/0.1% SDS at approximately 20-65° C. Alternatively, a substantially similar polypeptide may differ by at least 1, but less than 5, 10, 20, 50 or 100 amino acids from any of the sequences described herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:-

FIG. 1 shows maps of three embodiments of various different lentiviral vectors used to clone transgenes for an “Amalthea” feeder cell line (i.e. the feeder cell line according to the invention). FIG. 1A illustrates the lentiviral expression vector used to clone megaCD40L, FIG. 1B illustrates the lentiviral expression vector used to clone CD23B, and FIG. 1C illustrates the lentiviral expression vector used to clone the red fluorescent protein mCherry, by Gateway cloning. The custom vectors from Vector Builder contain selection markers for puromycin (PURO-for megaCD40L), blasticidin (BLAST-for CD23b), and neomycin (NEO-for mCherry). DNA fragments coding for the transgenes with the necessary Gateway cloning regions were manufactured by the GeneArt service of ThermoFisher Scientific.

FIG. 2 illustrates the feeder cells (i.e. MRC5, U2OS and LCL) megaCD40L expression in the cell medium. 10,000 cells were seeded in wells of a 96-well culture plate for each cell line, expressing megaCD40L following lentiviral transduction and puromycin selection. Two days later, supernatants were harvested and analysed by ELISA using Quantikine Human CD40 Ligand Immunoassay from Bio-Techne (Cat. No. DCDL40) according to manufacturer's instructions.

FIG. 3 demonstrates the ability of Amalthea feeders (i.e. the feeder cell line according to the invention) to improve B cell transformation efficiency. B cells were isolated and infected with GFP-expressing recombinant EBV. Infected cells were co-cultured with allogenic PBMC or Amalthea cells as feeders. Two days later flow cytometry was performed. Dead cells were stained with Draq7 (AbCam ab109202) and EBV infection was assessed by GFP expression. The potential for transformation of infected cells was assessed by expression of CD23 (13), through staining with anti-CD23-BV421 (Biolegend 338522).

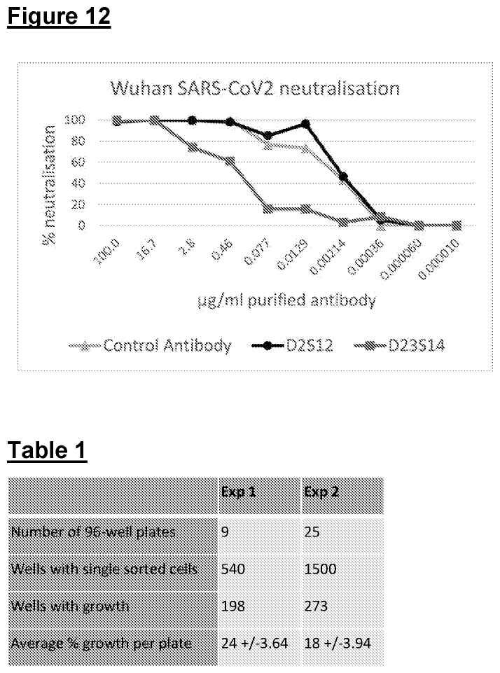

FIG. 4 illustrates that the presence of CD23b in feeder cells increases LCL outgrowth. B cells newly infected with recombinant EBV were placed in wells of 96-well culture plates that contained irradiated feeder cells with or without CD23b expression. On average, two B cells were placed in each well and after two weeks of cell culture, the number of wells with outgrowing LCLs was assessed by microscopy. The percentage of wells with outgrowing LCLs was determined for the respective feeders. Average percentages and standard deviation from two experiments are shown.

FIG. 5 shows that Amalthea feeders (i.e. the feeder cell line according to the invention) stably express mCherry. After G418 selection, Amalthea feeders were confirmed to be mCherry positive, and therefore, easily distinguishable by flow cytometry. The GFP channel was clear and available for GFP positive recombinant EBV-infected cells if needed.

FIG. 6 shows an indexed FACS of EBV-infected cells for single B cell cloning. A co-culture of EBV-infected LCL and Amalthea feeders was centrifuged once at 400g for two minutes and resuspended in RPMI with 5% FBS. Live, mCherry negative cells were single-cell sorted into 96-well plate wells. The upper panel shows the FACS gating strategy for the whole population, the lower panel shows the same gating strategy applied for indexed single-cell sorting in one 96-well plate. Cells were only sorted in the 60 inner wells of the plate.

FIG. 7 illustrates fluorescence microscopy of outgrowing monoclonal LCL cultures. GFP-expressing LCLs can be detected early after single-cell sorting (here at four days post-sort) and GFP expression is retained long-term (here 21 days) ensuring detection of growth. Amalthea mCherry positive feeders can also be visualised clearly and their state assessed during culturing.

FIG. 8 shows the FACS gating strategy used to sort for HIV Env-specific B cells. PBMC from blood of volunteers in a HIV vaccine trial were used to isolate live (Draq7 negative) B cells (CD19 positive) that were class-switched (IgM and IgD negative) and bound to HIV Env probes ConM GFP and/or ConS Scarlet.

FIG. 9 is a representation of data from ELISA on supernatants from monoclonal LCLs with specificity to HIV-1 Env immunogens. A) Human IgG production was verified for supernatants from 326 monoclonal cultures of LCL that originated from cells sorted for binding to HIV Env immunogens, ConM and ConS. Dots represent concentrations derived from antibody binding to anti-human antibodies and comparison to a known standard curve. Error bars are standard deviation from three replicates. B) After ELISA, to determine binding of supernatant antibody to ConM SOSIP HIV Env immunogen, the ratio of ConM binding to IgG concentration was calculated to assess affinity of MABs to ConM. A value of one means affinity to immunogen as high as the affinity of an anti-human antibody to IgG in the supernatants. C) Same as (B), but for ConS UFO HIV Env immunogen.

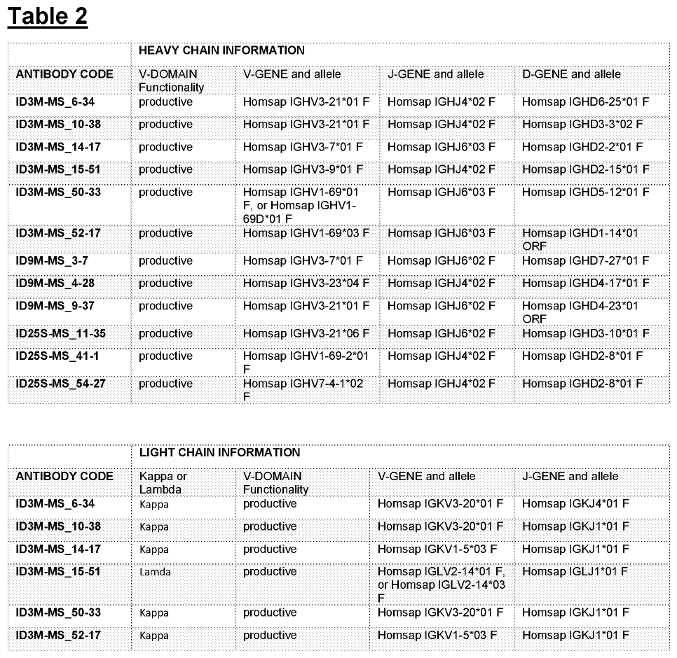

FIG. 10 illustrates the FACS gating strategy used to sort SARS-COV-2 spike-specific B cells. PBMCs from blood of COVID-19 convalescent patients were used to isolate live (Draq7 negative) B cells (CD19 positive) that were class-switched (IgM negative), IgA negative and can bind to the probe as seen by staining with an anti-MYC-AF488 antibody.

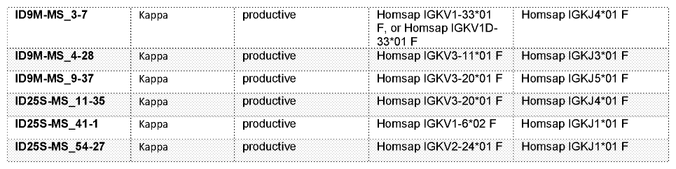

FIG. 11 shows pseudovirus neutralisation by supernatants of monoclonal LCL cultures. The capacity of antibodies in supernatants to neutralise a luciferase-expressing SARS-COV-2 pseudovirus and prevent infection of susceptible cells was assessed. The results of one 96-well plate are shown. Only the inner 60 wells of the plate were used for cultures. Each bar represents neutralisation level, relative to pseudovirus-only control, by a supernatant from the well of the corresponding position on the plate. Neutralisation level was determined by luciferase expression assays.

FIG. 12 shows pseudovirus neutralisation of purified antibodies against SARS-CoV2. Assay-verified MABs were purified and their neutralisation potency was studied by detailed neutralisation assays with serial dilutions as shown.

Table 1 summarises LCL outgrowth after indexed single-cell sorting. Wells were assessed for green LCL outgrowth with a fluorescent microscope after two weeks' culturing following indexed cell sorting. Outgrowth numbers and average percentages +/− standard deviation across different cell culture plates are shown for two independent experiments.

Table 2 summarises antibody information for 12 antibodies found to have high and broad affinity to HIV Env immunogens.

### EXAMPLES

As described throughout, there are several drawbacks associated with the current methods for isolating antigen-specific human monoclonal antibodies from B cells. Accordingly, the inventors set out to test whether their modified feeder cell line could be used to improve the efficiency and throughput of culturing and isolating monoclonal antibodies from B cells.

The inventors modified a feeder cell line to express megaCD40L, CD23b and mCherry fluorescent protein, using three lentiviral vectors (see FIG. 1). The inventors then tested whether the megaCD40L and CD23b expressed by the feeder cell line, could increase the transformation efficiency and outgrowth of the monoclonal antibody-producing B cells (Examples 2 and 3). The inventors further set out to test whether a fluorescent protein that differs from the fluorescent protein expressed by the B cell, could be used to distinguish the feeder cell line from the MAB producing B cells (Example 4). Finally, the inventors set out to demonstrate whether their feeder cell line could be used in methods of isolating MABs specific for HIV and SARS-COV-2 antigens (Examples 8 and 9).

### Materials and Methods

### Production of Recombinant EBV for Use in Infections of B Cells

This is as described in Reference (17). Briefly:
a) HEK293 EBV producer cells are grown to confluency in RPMI media with 10% FBS.
b) To produce recombinant EBV, producer cells are transfected with plasmids expressing transgenes that induce EBV lytic cycle and viral egress.
c) Recombinant EBV-containing supernatant is harvested.
d) Viral titre is determined by an assay that involves infection of lymphoma cells prone to infection using serial dilutions of harvested supernatant and counting GFP positive (green) cells. EBV stocks can be kept at 4° C. for >1 year.

### Lentiviral Transduction of Feeder Cells

Exogenous expression was achieved by lentiviral transduction of U2OS cells. A lentiviral expression vector with a puromycin selection cassette (FIG. 1A) was used to clone megaCD40L and to produce lentiviral particles. Puromycin at 1 μg/ml was used two days after transduction for selection. Expression of full length CD23b was achieved by lentiviral transduction of U2OS cells already expressing megaCD40L/Puro. A lentiviral expression vector with a blasticidin selection cassette was used (FIG. 1B) to clone CD23B and to produce viral particles. Blasticidin was added to the media two days after lentiviral transduction at a concentration of 10 μg/ml. The feeder cell line also expresses fluorescent protein mCherry for easy identification. This was facilitated by a custom lentiviral vector with a neomycin resistance gene (FIG. 1C). G418 was used at 250 μg/ml for selection two days after transduction.

### Culturing and Use of Amalthea Feeder Cells

a) Amalthea cells are adherent cells grown in RPMI media with 10% FBS.
b) To support LCL growth, on the previous day they are irradiated to arrest their growth and prevent them from taking over the culture. A ¹³⁷Cs γ-ray irradiator is used for a radiation dose of 30 Gy. Alternatively, an X-ray irradiator can be used. Alternatively, transient Mitomycin C treatment can be used.
c) After irradiation cells are counted and seeded into the cell culture container of choice. To form a confluent monolayer they are seeded at 8.3*10∧4 cells/cm².

### Method Protocol for MAB Isolation

a) FACS is used with an appropriate panel of markers to select live, class switched B cells that are specific for an antigen. Cells are kept at 4° C. at all times. This is a well-established process in the field and adapted to each specific antigen.
b) Sorted cells are infected with recombinant EBV by mixing the sorted cells with EBV-containing media at MOI 50 and incubated at 37° C. for 3 hours.
c) Cells are washed and resuspended with 0.5 ml RPMI media with 20% foetal bovine serum (FBS).
d) Resuspended cells are added to a well of a 48-well culture plate containing, in 0.5ml RPMI media with 20% FBS, feeder cells that have been irradiated and added to the well the previous day—enough time for the cells to settle, adhere and produce appropriate recombinant proteins for their feeder function. CpG ODN2006 (Invivogen tlrl-2006) is added to a final concentration of 2.5 μg/ml.
e) Infected cells are grown for 7 days in bulk.
f) After 7 days of growing infected cells (now LCL), FACS is used to deposit single, live, mCherry negative (not feeders) cells into wells of 96-well culture plates. Cell culture wells contain 2.510∧4 irradiated feeders in 50 μl RPMI with 20% FBS, prepared on the previous day. Each culture is now monoclonal.
g) 50 μl of fresh RPMI with 20% FBS and CpG ODN 2006 at a concentration of 5 μg/ml is added to each well, for a final concentration of 2.5 μg/ml
h) Cultures are fed twice a week by replacing 50 μl of media. For the first feed only, fresh media contains CpG ODN 2006. Feeding is performed using a pipetting robot (Integra Viaflo96).
i) Two weeks later monoclonal LCL cultures are assessed for growth by the presence of GFP positive EBV infected cells under a fluorescence microscope. Alternatively, a fluorescence plate reader can be used.
j) MAB-containing supernatants are harvested for assessment of MABs and cells from the outgrown cultures are harvested to determine the genetic sequence of MABs produced. If higher amounts of monoclonal antibodies are needed for molecular assays, monoclonal LCL cultures can be grown continuously and antibody-containing supernatant harvested as needed.

### Results and Discussion

### Example 1-Lentiviral Transduction of Feeder Cell Line

In order to clone transgenes for the “Amalthea” feeder cell line (i.e. the feeder cell line according to the invention), the inventors used three different lentiviral vectors, as illustrated in FIGS. 1A, 1B and 1C. The first and second lentiviral expression vectors (FIGS. 1A and 1B) were used to clone megaCD40L and CD23B, respectively, into the feeder cell line, to support the growth of the monoclonal antibody-producing B cells. The third lentiviral vector was used to clone the fluorescent protein mCherry, for easy identification of the feeder cell line.

### Example 2-Amalthea Feeders Increase Transformation Efficiency

To test B cell transformation efficiency with the Amalthea feeders (i.e. the feeder cell line according to the invention), B cells were isolated from human blood samples using Miltenyi B cell isolation kit (130-091-151). These were infected with recombinant EBV by mixing them with virus stock solution at multiplicity of infection (MOI) of 50 and incubating them for three hours at 37° C. with 5% CO₂, and then washing them with RPMI media and resuspending in RPMI supplemented with 20% fetal bovine serum (FBS). Half the infected cells were placed in a well of a 24-well plate with 2.5*10∧5 irradiated (30Gy) PBMC acting as feeders and the other half in a well with 1.75*10∧5 irradiated (30Gy) Amalthea cells as feeders. CpG ODN2006 (Invivogen tlrl-2006) was added to a final concentration of 2.5 μg/ml in all wells. Cyclosporin A was added to the well with allogenic PBMC at 1 μg/ml. Feeder cells were irradiated and seeded the day before infection.

Two days post-infection, flow cytometry was performed to determine the percentage of B cells that were infected (GFP positive) and activated (CD23 positive). At two days post-infection, cells are activated (13) but proliferation has not started (18). There was more than a 4-fold increase in B cells that were infected and activated on their way to becoming LCLs with the Amalthea feeders, as illustrated in FIG. 3. Specifically, only 15% of live cells were infected and activated when supported by PBMC feeders, compared to 62.8% of live cells being infected and activated when supported by Amalthea feeders.

### Example 3-Amalthea Feeders Increase LCL Outgrowth by Expression of CD23b

Two 96-well culture plates were prepared by seeding 2.5*10∧4 irradiated feeders into each well. The feeders for one culture plate were U2OS cells expressing exogenously megaCD40L and mCherry (Amalthea precursor cells before introduction of lentivirus for CD23b expression). The feeders for the other culture plate were Amalthea cells, additionally transduced with CD23b lentivirus. On the following day, B cells were isolated and infected with recombinant EBV as described above. Live cells were counted and serial dilution in RPMI media with 20% FBS was performed so that on average, two live cells were placed in each well of the 96-well culture plates containing feeders with or without CD23b expression. RPMI media was supplemented with CpG ODN2006 as described above. Cultures were grown for two weeks, feeding on days 2, 6 and 12 by replacing 50 μl media, in the first feed again supplemented with CpG ODN2006. On day 14, LCL growth was assessed by fluorescent microscopy and the percentage of wells with growth was plotted (FIG. 4). Across two experiments, there was approximately 20% increase in the number of outgrowing LCL cultures when the Amalthea feeders expressing CD23b were used.

### Example 4-Amalthea Feeders are Easily Distinguishable From the MAB Producing B Cells

Amalthea feeders are stably transduced with a lentivirus for mCherry and neomycin resistance gene expression. G418 at 2 mg/ml was used for selection and flow cytometry was used to confirm that virtually all cells are mCherry positive (FIG. 5). mCherry expression in the Amalthea cells makes them easily distinguishable from mCherry negative LCL, and therefore, it is not necessary to stain the new LCLs before FACS for single B cell cloning, even if a non-recombinant, non-GFP-expressing EBV is used. This is significantly advantageous, given that additional staining steps would have caused loss of cells of interest.

### Example 5-Amalthea Feeders Can be Used Without Irradiation

Amalthea feeders do not express the gene for hygromycin resistance, as LCLs infected with recombinant EBV do. This means that hygromycin can be used to kill off the feeder cells in a co-culture when they are no longer needed, without affecting antibody-producing hygromycin resistant LCL cells, when recombinant EBV is used. This makes the platform flexible enough to potentially be used when an irradiator is not available to arrest feeder cells and it is not desirable to use a drug treatment.

### Example 6-Amalthea Feeders Facilitate Confirmed Single B Cell Cloning

Indexed single-cell FACS of B cells into wells of 96-well plates was used for the first time for B cell (LCL) culture cloning. Antigen specific, class switched B cells were infected with recombinant EBV and cultured in bulk for the first seven days on irradiated (30 Gy) Amalthea feeders that were seeded the previous day at 1.75*10∧5 cells in a well of a 24-well plate. After seven days, the cells of the early bulk culture (new LCL and Amalthea feeders) were single-cell sorted into 96-well plate wells. Live, mCherry negative cells were single-cell sorted into the inner 60 wells (FIG. 6). The outermost wells were filled with sterile PBS to prevent evaporation from the culture wells. Sorted cells were cultured in the 96-well plate format for two weeks as described above and then wells were assessed for LCL growth. In two independent experiments, 540 and 1500 cells were sorted in individual wells with growth observed in 198 and 273 wells, respectively (Table 1).

### Example 7-GFP-Expressing LCL Can be Easily Visualised for Outgrowth Assessment

Single-cell sorted cultures can be followed and assessed throughout the outgrowth period (FIG. 7), facilitating easy and certain identification of outgrowing cultures and allowing forward planning without culture loss. The platform has the potential for automated outgrowth detection with fluorescence plate readers that can massively increase throughput potential.

### Example 8-MABs Against HIV Envelope Protein

The following examples demonstrate that the invention is effective at isolating useful MABs. The method is very high throughput and after three weeks supernatants containing antigen specific monoclonal antibodies are available for multiple assays, here shown for ELISA and neutralisation assays. This means that only assay-verified MABs will be chosen for sequence analysis and downstream applications, reducing cost and effort. Uniquely for this invention, this is done straight from B cell cultures that are verified monoclonal from the start.

Blood samples were obtained from volunteers in an HIV vaccine trial. The immunogens used to vaccinate the volunteers were based on a HIV-1 envelope (Env) glycoprotein consensus sequence. Two immunogen variants were used, termed ConM SOSIP and ConS UFO, representing two different strategies of stabilising soluble immunogens in a native-like conformation for HIV Env.

PBMC were isolated by gradient centrifugation and live, class-switched B cells specific for the immunogens were sorted by FACS. To sort for immunogen-specific B cells, two probes were produced, comprising the protein sequence of either ConM SOSIP or ConS UFO, fused to either superfolder GFP (sfGFP) or mScarlet-I fluorescent protein, respectively (ConM-GFP and ConS-Scarlet). B cells specific for ConM-GFP and/or ConS-Scarlet were sorted as shown in FIG. 8.

Sorted cells were infected with recombinant EBV and co-cultured with Amalthea feeders for seven days, as described above, to produce proliferating LCLs from the antigen-specific B cells. LCL cells were then single-cell sorted into 96-well plates and co-cultured with Amalthea feeders as described above, using the gating strategy shown in FIG. 6.

326 monoclonal LCL cultures grew out and supernatants and cells were harvested for all. The supernatants were tested by ELISA for human IgG antibody presence, and all were found to be positive with concentrations ranging between 100-10000 ng/ml and most with a concentration of 1-2 μg/ml (FIG. 9A). Separate ELISAs on the same supernatants were performed to assess specificity to ConM SOSIP and ConS UFO and the vast majority exhibited binding to at least one immunogen. The ratio of ConM SOSIP or ConS UFO binding to IgG concentration in the supernatants gave a readout of antibody affinity for each immunogen (FIG. 9B and 9C). The information on affinity for each monoclonal antibody produced helped to choose the 12 best monoclonal antibodies for affinity level and breadth and determine their sequence for further study (Table 2). The antibody sequences were determined with techniques broadly used in the field. Briefly, harvested LCL cells were frozen at −80° C. in a lysis buffer with 0.01 M Tris pH8.0 and Ribolock RNase inhibitor (ThermoFisher EO0381). cDNA was produced from the cells' mRNA by reverse transcription with SuperScript IV Reverse Transcriptase (Thermo Fisher 18090200) and random hexamers (ThermFisher N8080127). PCR amplification with primer pools specific for human antibodies (4) and Sanger sequencing of the PCR products revealed the MAB sequences.

### Example 9-Neutralising MABs Against SARS COV2

Blood samples were obtained from convalescent COVID-19 patients and PBMC were isolated by gradient centrifugation. A soluble SARS-COV-2 MYC-tagged protein was produced (19) and was used as a probe to sort for antigen specific B cells (FIG. 10). Sorted SARS-COV-2 specific B cells were infected by recombinant EBV and monoclonal LCL cultures were produced as described above. Supernatants of monoclonal cultures were harvested from 75 96-well plates two weeks after single cells were sorted in their wells. The supernatants were used directly for standard neutralisation assays of a SARS-COV-2 pseudovirus as described in (19) (FIG. 11). Several neutralising supernatants were identified with varied neutralisation potency. For the best neutralisers, the antibody sequence was determined as described in Example 8 and more antibody was produced and purified for more detailed studies. As an example, FIG. 12 shows a comprehensive neutralisation study for two of the inventors' isolated and purified antibodies in comparison with a control antibody isolated by others. The inventors' D2S15 MAB neutralises better than the other antibodies tested at higher dilution.

### Conclusions

As illustrated throughout the Examples, the inventors surprisingly discovered that compared to the classic prior art EBV method, their Amalthea feeder cell line, increases EBV-mediated transformation/immortalisation efficiency of B cells by more than 4-fold. In particular, the inventors have identified that modifying a feeder cell line to express mega CD40L and/or CD23, results in a feeder cell line that can significantly increase the number of outgrowing monoclonal antibody-producing B cells.

Additionally, with the inventors' new feeder cell line, it is now possible to support monoclonal outgrowth of EBV-infected cells for the first time without subcloning steps, saving several weeks compared to the classic EBV method for monoclonal antibody isolation and greatly increasing efficiency. Furthermore, the resulting monoclonal cultures' supernatants contain antibody that can be assessed without the need for molecular cloning of the antibody sequence, which is required for the method most favoured currently. This brings down the cost per antibody assessed 50-to 100-fold, relative to the single-cell molecular cloning method currently preferred.

Finally, by modifying the feeder cell line to express a fluorescent protein that is not expressed by the B cell, the inventors have demonstrated that it is possible to distinguish the feeder cells from the B cells, without any additional staining steps that would result in the loss of cells of interest.

### References

1. Steinitz, M., Klein, G., Koskimies, S. and Makel, O. (1977) EB virus-induced B lymphocyte cell lines producing specific antibody. Nature, 269, 420-422.
2. Traggiai, E., Becker, S., Subbarao, K., Kolesnikova, L., Uematsu, Y., Gismondo, M.R., Murphy, B.R., Rappuoli, R. and Lanzavecchia, A. (2004) An efficient method to make human monoclonal antibodies from memory B cells: potent neutralization of SARS coronavirus. Nat Med, 10, 871-875.
3. Steinitz, M. (2014) Production of human monoclonal antibodies by the epstein-barr virus method. Methods Mol Biol, 1060, 111-122.
4. Tiller, T., Meffre, E., Yurasov, S., Tsuiji, M., Nussenzweig, M.C. and Wardemann, H. (2008) Efficient generation of monoclonal antibodies from single human B cells by single cell RT-PCR and expression vector cloning. J Immunol Methods, 329, 112-124.
5. Ehlers, A.M., den Hartog Jager, C.F., Kardol-Hoefnagel, T., Katsburg, M.M.D., Knulst, A.C. and Otten, H.G. (2021) Comparison of Two Strategies to Generate Antigen-Specific Human Monoclonal Antibodies: Which Method to Choose for Which Purpose? Front Immunol, 12, 660037.
6. Holler, N., Tardivel, A., Kovacsovics-Bankowski, M., Hertig, S., Gaide, O., Martinon, F., Tinel, A., Deperthes, D., Calderara, S., Schulthess, T. et al. (2003) Two adjacent trimeric Fas ligands are required for Fas signaling and formation of a death-inducing signaling complex. Mol Cell Biol, 23, 1428-1440.
7. Stone, G.W., Barzee, S., Snarsky, V., Kee, K., Spina, C.A., Yu, X.F. and Kornbluth, R.S. (2006) Multimeric soluble CD40 ligand and GITR ligand as adjuvants for human immunodeficiency virus DNA vaccines. J Virol, 80, 1762-1772.
8. Guo, R., Zhang, Y., Teng, M., Jiang, C., Schineller, M., Zhao, B., Doench, J.G., O'Reilly, R.J., Cesarman, E., Giulino-Roth, L. et al. (2020) DNA methylation enzymes and PRC1 restrict B-cell Epstein-Barr virus oncoprotein expression. Nat Microbiol, 5, 1051-1063.
9. Wood, C.D., Veenstra, H., Khasnis, S., Gunnell, A., Webb, H.M., Shannon-Lowe, C., Andrews, S., Osborne, C.S. and West, M.J. (2016) MYC activation and BCL2L11 silencing by a tumour virus through the large-scale reconfiguration of enhancer-promoter hubs. Elife, 5.
10. Swendeman, S. and Thorley-Lawson, D.A. (1987) The activation antigen BLAST-2, when shed, is an autocrine BCGF for normal and transformed B cells. EMBO J, 6, 1637-1642.
11. Cairns, J.A. and Gordon, J. (1990) Intact, 45-kDa (membrane) form of CD23 is consistently mitogenic for normal and transformed B lymphoblasts. Eur J Immunol, 20, 539-543.
12. Azim, T., Allday, M.J. and Crawford, D.H. (1990) Immortalization of Epstein-Barr virus-infected CD23-negative B lymphocytes by the addition of B cell growth factor. J Gen Virol, 71 (Pt 3), 665-671.
13. Thorley-Lawson, D.A. and Mann, K.P. (1985) Early events in Epstein-Barr virus infection provide a model for B cell activation. J Exp Med, 162, 45-59.
14. Delecluse, H.J. and Hammerschmidt, W. (2000) The genetic approach to the Epstein-Barr virus: from basic virology to gene therapy. Mol Pathol, 53, 270-279.
15. Anderton, E., Yee, J., Smith, P., Crook, T., White, R.E. and Allday, M.J. (2008) Two Epstein-Barr virus (EBV) oncoproteins cooperate to repress expression of the proapoptotic tumour-suppressor Bim: clues to the pathogenesis of Burkitt's lymphoma. Oncogene, 27, 421-433.
16. White, R.E., Groves, I.J., Turro, E., Yee, J., Kremmer, E. and Allday, M.J. (2010) Extensive co-operation between the Epstein-Barr virus EBNA3 proteins in the manipulation of host gene expression and epigenetic chromatin modification. PLoS One, 5, e13979.
17. Tierney, R.J., Kao, K.Y., Nagra, J.K. and Rickinson, A.B. (2011) Epstein-Barr virus BamHI W repeat number limits EBNA2/EBNA-LP coexpression in newly infected B cells and the efficiency of B-cell transformation: a rationale for the multiple W repeats in wild-type virus strains. J Virol, 85, 12362-12375.
18. Nikitin, P.A., Yan, C.M., Forte, E., Bocedi, A., Tourigny, J.P., White, R.E., Allday, M.J., Patel, A., Dave, S.S., Kim, W. et al. (2010) An ATM/Chk2-mediated DNA damage-responsive signaling pathway suppresses Epstein-Barr virus transformation of primary human B cells. Cell Host Microbe, 8, 510-522.
19. Mckay, P.F., Hu, K., Blakney, A.K., Samnuan, K., Brown, J.C., Penn, R., Zhou, J., Bouton, C.R., Rogers, P., Polra, K. et al. (2020) Self-amplifying RNA SARS-COV-2 lipid nanoparticle vaccine candidate induces high neutralizing antibody titers in mice. Nat Commun, 11, 3523.

1. A feeder cell line for culturing a monoclonal antibody-producing B cell, the feeder cell line expressing:
mega CD40 ligand (mega CD40L), or a variant or fragment thereof;
CD23, or a variant or fragment thereof; and/or
a fluorescent protein that is not expressed by the B cell.

2. The feeder cell line according to claim 1, wherein the feeder cell line is selected from a group consisting of an osteosarcoma cell line, a mesenchymal cell line, an epithelial cell line, a lymphoblastoid cell line, a neuronal cell line and an endothelial cell line, preferably wherein the feeder cell line is an osteosarcoma cell line.

3. The feeder cell line according to either claim 1 or claim 2, wherein the feeder cell line is selected from a group consisting of U2OS, MRC5, lymphoblastoid cell line (LCL), H1299, MCF7, HEK293, 3T3, Caco-2, and HeLa, preferably wherein the feeder cell line is U2OS.

4. The feeder cell line according to any one of the preceding claims, wherein the feeder cell line is irradiated, preferably wherein the feeder cell line is irradiated using a ¹³⁷Cs γ-ray irradiator or an X-ray irradiator, or wherein the feeder cell line is treated transiently with Mitomycin C.

5. The feeder cell line according to any one of the preceding claims, wherein the mega CD40L, or a variant or fragment thereof comprises an amino acid sequence substantially as set out in SEQ ID No: 1, or a fragment or variant thereof, and/or wherein the mega CD40L, or a variant or fragment thereof is encoded by a nucleotide sequence substantially as set out in SEQ ID No: 2, or a fragment or variant thereof.

6. The feeder cell line according to any one of the preceding claims, wherein the feeder cell line expresses
(i) at least 1 ng/ml, at least 2 ng/ml, at least 3 ng/ml, at least 4 ng/ml, or at least 5 ng/ml of mega CD40L, or a variant or fragment thereof, in a cell culture media;
(ii) at least 10 ng/ml, at least 20 ng/ml, at least 30 ng/ml, at least 40 ng/ml, or at least 50ng/ml of mega CD40L, or a variant or fragment thereof, in a cell culture media;
(iii) at least 51 ng/ml of mega CD40L, or a variant or fragment thereof, in a cell culture media; and/or
(iv) at least 101 ng/ml of mega CD40L, or a variant or fragment thereof, in a cell culture media.

7. The feeder cell line according to any one of the preceding claims, wherein the CD23 or fragment or variant thereof, expressed by the feeder cell line, is CD23a or CD23b.

8. The feeder cell line according to any one of the preceding claims, wherein the feeder cell line expresses CD23b, preferably wherein CD23b comprises an amino acid sequence substantially as set out in SEQ ID No: 3, or a variant or fragment thereof, and/or wherein CD23b is encoded by a nucleotide sequence substantially as set out in SEQ ID No: 4, or a fragment or variant thereof.

9. The feeder cell line according to any one of claims 1-7, wherein the feeder cell line expresses CD23a, preferably wherein CD23a comprises an amino acid sequence substantially as set out in SEQ ID No: 5, or a variant or fragment thereof, and/or wherein CD23a is encoded by a nucleotide sequence substantially as set out in SEQ ID No: 6, or a fragment or variant thereof.

10. The feeder cell line according to any one of the preceding claims, wherein the feeder cell line expresses a first fluorescent protein, and the B cell expresses a second fluorescent protein, wherein the first and second fluorescent proteins are different.

11. The feeder cell line according to any one of the preceding claims, wherein the B cell expresses enhanced green fluorescent protein (GFP).

12. The feeder cell line according to claim 11, wherein the enhanced GFP comprises an amino acid sequence substantially as set out in SEQ ID No: 8, or a fragment or variant thereof, and/or wherein the enhanced GFP is encoded by a nucleotide sequence substantially as set out in SEQ ID No: 9, or a fragment or variant thereof.

13. The feeder cell line according to any one of the preceding claims, wherein the feeder cell line expresses mCherry.

14. The feeder cell line according to claim 13, wherein mCherry comprises an amino acid sequence substantially as set out in SEQ ID No: 10, or a fragment or variant thereof, and/or wherein mCherry is encoded by a nucleotide sequence substantially as set out in SEQ ID No: 11, or a fragment or variant thereof.

15. The feeder cell line according to any one of the preceding claims, wherein the monoclonal antibody-producing B cell is cultured using Epstein-Barr virus (EBV), preferably wherein the EBV is recombinant EBV.

16. The feeder cell line according to claim 15, wherein the recombinant EBV expresses a drug selection marker, preferably wherein the recombinant EBV expresses a hygromycin resistance gene.

17. The feeder cell line according to claim 16, wherein the hygromycin resistance gene comprises a nucleotide sequence substantially as set out in SEQ ID No: 7, or a fragment or variant thereof.

18. Use of the feeder cell line according to any one of claims 1-17, in culturing a monoclonal antibody-producing B cell.

19. Use of the feeder cell line according to any one of claims 1-17, in isolating a monoclonal antibody-producing B cell.

20. A method of culturing a monoclonal antibody-producing B cell, the method comprising:
(i) contacting a B cell with the feeder cell line according to any one of claims 1-17; and
(ii) culturing the B cell and the feeder cell line under conditions to support the growth of the monoclonal antibody-producing B cell.

21. A method of isolating a monoclonal antibody-producing B cell from a cell culture media, the method comprising:
(i) contacting a B cell with the feeder cell line according to any one of claims 1-17; and
(ii) identifying the feeder cell line expressing the fluorescent protein that is not expressed by the B cell, to allow isolation of the monoclonal antibody-producing B cell.

22. A method of isolating a monoclonal antibody-producing B cell from a cell culture media, the method comprising:
(i) contacting a B cell with the feeder cell line according to either claim 16 or 17; and
(ii) culturing the B cell and the feeder cell line in the presence of the drug selection marker to allow isolation of the monoclonal antibody-producing B cell.

23. The method according to any one of claims 20-22, wherein the method further comprises isolating a monoclonal antibody from the monoclonal antibody-producing B cell.

24. A method of isolating a monoclonal antibody from a monoclonal antibody-producing B cell, the method comprising:
(i) contacting a B cell with the feeder cell line according to any one of claims 1-17;
(ii) culturing the B cell and the feeder cell line under conditions to support the growth of the monoclonal antibody-producing B cell; and
(iii) isolating a monoclonal antibody from the monoclonal antibody-producing B cell.

25. The method according to any one of claims 20 to 24, further comprising isolating a B cell from a sample obtained from a subject, preferably wherein the B cell is specific for an antigen of interest.

26. The method according to any one of claims 20 to 25, further comprising contacting the B cell with a CpG oligonucleotide, preferably wherein the CpG oligonucleotide is a nuclease resistant phosphorothioate oligonucleotide.

27. The method according to claim 26, wherein the CpG oligonucleotide comprises a nucleotide sequence substantially as set out in SEQ ID No: 12, or a fragment or variant thereof.

28. A lentiviral vector substantially as illustrated in FIG. 1A, 1B or 1C.

29. A kit for culturing a monoclonal antibody-producing B cell, the kit comprising a feeder cell line expressing:
(a) mega CD40 ligand (mega CD40L), or a variant or fragment thereof;
(b) CD23, or a variant or fragment thereof; and/or
(c) a fluorescent protein that is not expressed by the B cell.

30. The kit according to claim 29, for performing the method of any one of claims 20-27, and/or using the feeder cell line according to any one of claims 1-17.


SEQUENCE LISTING (12 sequences)

SEQ ID NO: 1 - protein, 273 aa, synthetic construct
MKANLLVLLCALAAADADYKDDDDKGPGQVQLHEDDVTTTEELAPALVPPPKGTCAGWMAGIPGHPGHNGTPGRDGRDGTPGEKGEKGDAGLLGPKGETGDVGMTGAEGPRGFPGTPGRKGEPGELQGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - DNA, 822 bp, synthetic construct
  source 1..822: mol_type = other DNA
atgaaggccaacctgctggtgctgctgtgtgctctggctgccgccgatgccgactacaaggacgacgatgacaaaggccctggacaggtccagctgcacgaggatgatgtgaccaccaccgaagaactggcccctgctcttgtgcctcctccaaagggaacatgtgccggctggatggctggaatccctggacacccaggccacaatggcacacctggcagagatggaagagatggcaccccaggcgagaagggcgaaaaaggcgacgctggactgctgggacctaaaggcgaaactggcgacgtgggaatgacaggcgctgagggccctagaggctttcctggaacacctggaagaaagggcgagcctggcgaactgcagggcgatcagaatcctcagattgccgctcacgtgatcagcgaggccagcagcaagacaacaagcgtgctgcagtgggccgagaaggggtactacaccatgagcaacaacctggtcaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagctccgccaaaccttgtggccagcagtctattcacctcggcggagtgtttgagctgcagcctggcgctagcgtgttcgtgaatgtgacagaccctagccaggtgtcccacggcaccggctttacatctttcggcctgctgaagctgtga

SEQ ID NO: 3 - protein, 320 aa, Homo sapiens
MNPPSQEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 4 - DNA, 963 bp, Homo sapiens
  source 1..963: mol_type = genomic DNA
atgaatcctccaagccaagagatcgaggaactgccccgcagacggtgctgtagaagaggcacacagatcgtgctgctgggccttgtgacagctgctctgtgggctggactgctgacactgctgctgctgtggcactgggataccacacagagcctgaagcagctggaagaaagggccgccagaaacgtgtcccaggtgtccaagaacctggaaagccaccacggcgaccagatggcccagaagtctcagagcacccagatcagccaagagcttgaagaactgagagccgagcagcagcggctgaagtcccaagatctggaactgagctggaacctgaacggactgcaggccgatctgagcagcttcaagtctcaagagctgaacgagagaaacgaggccagcgacctgctggaacggctgagagaagaagtgaccaagctgcggatggaactgcaggtttccagcggcttcgtgtgcaacacatgccccgagaagtggatcaacttccagcggaagtgctactacttcggcaagggcaccaagcagtgggtgcacgccagatacgcctgcgacgatatggaaggccagctggtgtccattcacagccccgaggaacaggacttcctgaccaaacacgccagccacaccggctcttggatcggactgagaaacctggacctgaagggcgagttcatctgggtggacggcagccacgtggactactctaattgggctcctggcgagcccaccagcagatctcaaggcgaggattgcgtgatgatgagaggcagcggcagatggaacgacgccttctgcgatagaaagctcggcgcctgggtttgcgacagactggccacatgtacacctccagcctctgagggaagcgccgagtctatgggccctgactctagacccgatcctgacggcagactgcctacaccttctgctcctctgcacagctga

SEQ ID NO: 5 - protein, 321 aa, Homo sapiens
MEEGQYSEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 6 - DNA, 966 bp, Homo sapiens
  source 1..966: mol_type = genomic DNA
atggaagagggccagtacagcgagatcgaggaactgcctcggcggagatgctgtagaagaggcacacagatcgtgctgctgggccttgtgacagctgctctgtgggctggactgctgacactgctgctgctgtggcactgggataccacacagagcctgaagcagctggaagaaagggccgccagaaacgtgtcccaggtgtccaagaacctggaaagccaccacggcgaccagatggcccagaagtctcagagcacccagatcagccaagagcttgaggaactgagagccgagcagcagagactgaagtcccaggacctggaactgagctggaacctgaatggactgcaggccgacctgagcagcttcaagtcccaagagctgaacgagagaaacgaggccagcgacctgctggaacggctgagagaagaagtgaccaagctgcggatggaactgcaggtttccagcggcttcgtgtgcaacacatgccccgagaagtggatcaacttccagcggaagtgctactacttcggcaagggcaccaagcagtgggtgcacgccagatacgcctgcgacgatatggaaggccagctggtgtccattcacagccccgaggaacaggacttcctgaccaaacacgccagccacaccggctcttggatcggcctgagaaacctggatctgaagggcgagttcatctgggtggacggcagccacgtggactactctaattgggctcctggcgagcccaccagcagatctcaaggcgaggattgcgtgatgatgagaggcagcggcagatggaacgacgccttctgcgatagaaagctcggcgcctgggtttgcgacagactggccacatgtacacctccagcctctgagggaagcgccgagtctatgggccctgactctagacccgatcctgacggcagactgcctacaccttctgctcctctgcacagctga

SEQ ID NO: 7 - DNA, 1026 bp, Human gammaherpesvirus 4
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 8 - protein, 265 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSAVDGTAGPGSTGSR

SEQ ID NO: 9 - DNA, 798 bp, synthetic construct
  source 1..798: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataa

SEQ ID NO: 10 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 11 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 12 - DNA, 24 bp, synthetic construct
  source 1..24: mol_type = other DNA
tcgtcgttttgtcgttttgtcgtt